(19) United States
(12) Patent Application Publication
(10) Pub. No.: US 2023/0389584 A1
(43) Pub. Date: Dec. 7, 2023

(54) OLIVE-DERIVED COMPOSITIONS

(71) Applicant: MEDIAKOS GmbH, Berlin (DE)

(72) Inventors: Martha-Spyridoula Katsarou, Nikea (GR); Prokopios Magiatis, Ambelakia (GR); Eleni Melliou, Ambelakia (GR); Nikolaos Drakoulis, Psychiko (GR)

(21) Appl. No.: 18/033,761

(22) PCT Filed: Oct. 26, 2021
(86) PCT No.: PCT/EP2021/079613

§ 371 Date: Apr. 25, 2023

(30) Foreign Application Priority Data
Oct. 26, 2020 (EP) 20203915.2

(51) Int. Cl. A23L 19/00; A23L 33/105; C11B 1/06

(52) U.S. Cl. CPC A23L 19/09; A23L 33/105; C11B 1/06

(57) ABSTRACT

The present invention relates to a method of producing at least one composition, said method comprising the following steps: (a) preparing a paste from flesh of fermented Kalamon olives; (b) subjecting said paste to a separation process yielding oil, a semisolid fraction, and an aqueous phase, said separation process preferably being centrifugation; and (c) performing one, two or three of the following: (i) harvesting said oil, thereby obtaining a first composition; (ii) drying said semisolid fraction, thereby obtaining a second composition; and (iii) harvesting said aqueous phase, thereby obtaining a third composition.

The present invention relates to a method of producing at least one composition, said method comprising the following steps: (a) preparing a paste from flesh of fermented Kalamon olives; (b) subjecting said paste to a separation process yielding oil, a semisolid fraction, and an aqueous phase, said separation process preferably being centrifugation; and (c) performing one, two or three of the following: (i) harvesting said oil, thereby obtaining a first composition; (ii) drying said semisolid fraction, thereby obtaining a second composition; and (iii) harvesting said aqueous phase, thereby obtaining a third composition.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Olives and olive oil have been described to modulate lipid metabolism. The influence of olive species, the geographic region of origin and treatment of olives have not yet received sufficient attention when it comes to optimizing the beneficial effects of olive-derived products. Furthermore, the prior art fails to teach which specific fractions of blood cholesterol may be beneficially modulated. In addition, the prior art fails to consider pre-treatment of olives prior to obtaining any products from olives.

While certain individuals may appreciate the taste of olives, this does not apply to all individuals who might benefit from the advantageous properties of a frequent consumption of olives.

EP 2238840 A1 describes a method of producing extracted olive oil. Said method involves the deliberate use or addition of olive leaves to the olives to be processed.

This is described as a means of improving the cosmetic properties of the obtained olive oil.

The above described state of the art as well as deficiencies thereof have been recognized by the present inventors.

The technical problem underlying the present invention can be seen in the provision of improved means and methods of modulating lipid metabolism.

This technical problem has been solved by the enclosed claims.

Accordingly, in the first aspect, the present invention relates to a method of producing at least one composition, said method comprising the following steps: (a) preparing a paste from flesh of fermented Kalamon olives; (b) subjecting said paste to a separation process yielding oil, a semisolid fraction, and an aqueous phase, said separation process preferably being centrifugation; and (c) performing one, two or three of the following: (i) harvesting said oil, thereby obtaining a first composition; (ii) drying said semisolid fraction, thereby obtaining a second composition; and (iii) harvesting said aqueous phase, thereby obtaining a third composition.

Said second composition is also referred to as composition S (for semisolid) in the following. Said third composition is also referred to as composition A (for aqueous) in the following. Compositions A and S are preferred compositions, and composition A is most preferred; see also the preferred preparation procedure disclosed further below.

Preferably, no further processing is applied to and/or no further use is made of said oil or first composition in the context of the present invention.

In other words, and related to the first aspect, the invention relates to a method of producing at least one composition, said method comprising the following steps: (a) preparing a paste from flesh of fermented Kalamon olives; (b) subjecting said paste to a separation process yielding oil, a semisolid fraction, and an aqueous phase, said separation process preferably being centrifugation; and (c) performing (i) harvesting said aqueous phase, thereby obtaining a composition A; and optionally (ii) drying said semisolid fraction, thereby obtaining a composition S.

Kalamon olives are a variety of olives. They are also referred to as Kalamata olives. The systematic name is Olea europaea L., cv. Kalamata.

Olive flesh of fermented Kalamon olives is transformed into a paste in accordance with step (a) of the method of the first aspect. For this purpose, preferably a hammer mill is used.

Said olive flesh is free or essentially free of olives leaves. In particular, said method of the first aspect does not involve a step of adding olive leaves.

As is apparent from further aspects of the invention as disclosed further below, compositions obtained by the method of the first aspect are for ingestion and the manufacture of a food supplement. They are preferably not for cosmetic purposes.

The separation process in accordance with step (b) is preferably implemented by a centrifugation procedure on a three-phase decanter. The paste yields oil, a semisolid fraction, and an aqueous phase which may also be referred to as first, second and third fraction, respectively.

In accordance with step (c), said semisolid fraction may be subjected to drying. Preferably, drying is done by a two-step dehydration procedure comprising the first step of air drying and a second step of machine-driven drying such as flow bed drying or lyophilisation.

Harvesting of oil and/or aqueous phase is straightforward and can be done, e.g. by decanting and/or pumping.

A commonly known product obtained from olives is olive oil. Of note, the requirement in accordance with the invention of the olives being fermented is not routinely met in the preparation of olive oil. This distinction renders the fractions or compositions obtained from fermented Kalamon olives different from common olive products.

In particular, and having regard to said first composition or said oil in accordance with the invention, using fermented olives as starting material leads to an increase in contents of hydroxytyrosol and tyrosol. Common olive oil contains esters of these compounds such as oleacein and oleocanthal. Fermentation of the olives prior to processing in accordance with the invention entails hydrolysis of these compounds and sets free hydroxytyrosol and tyrosol. This is one of the preferred features which distinguishes the oil and the first composition obtained by the method of the invention from common olive oil.

Having regard to the aqueous phase and the third composition of the invention, it is of note that fermented olives have an elevated content of lactic acid. This renders the compositions of the invention, especially the third composition, distinct from aqueous extracts of non-fermented olives.

In a preferred embodiment of the method of the first aspect, said method further comprises (ba) washing, preferably a plurality of times such as three times, said semisolid fraction obtained in (b) with water, thereafter combining said water with said aqueous phase obtained in (b), thereby obtaining a washed semisolid fraction and a combined aqueous phase; and/or (ca) filtrating said oil obtained in (c)(i), thereby obtaining a filtrated first composition.

It is understood that at the point in time when water that has been used for the washing step(s) is subsequently combined, it comprises material originally contained in said semisolid fraction, in particular such material which is amenable to aqueous extraction. In other words, said water is indeed an aqueous extract of said semisolid fraction.

Preferably said water, i.e., said aqueous extract of said semisolid fraction is combined with the aqueous phase obtained in step (b) of the method of the first aspect. As a consequence, a composition A is obtained which combines the aqueous phase of said separation process with the aqueous extract of said semisolid fraction, said semisolid fraction in turn also being a result of the initial separation process in accordance with the first aspect.

On the other hand, it is not compulsory to combine the above defined aqueous extract with the initial aqueous phase. In such a case, a composition A1 is obtained which is said initial aqueous phase, and a composition A2 which consists of an aqueous extract of said semisolid phase. Either composition (A1 and A2 or one of them) may independently be subsequently processed to yield a capsule; see below.

A particularly preferred implementation of the above preferred embodiment provides for (ba) washing, preferably a plurality of times such as three times, said semisolid fraction obtained in (b) with water, thereafter combining said water with said aqueous phase obtained in (b), thereby obtaining a washed and dried composition S in step (c), and a combined aqueous phase yielding composition A of step (c).

It is understood that washing said semisolid fraction entails the need for separating the water used for washing from said semisolid fraction which has undergone washing. This separating is preferably accomplished by filtrating.

Preferably, said filtrating, be it of the oil of or the washed semisolid fraction, is implemented by means of a filter press. This removes any insoluble material. In case of filtrating said oil, the residue on the filter is preferably discarded. In case of washing said semisolid fraction, any residue on the filter is preferably retained and combined to give rise to the washed semisolid fraction.

Repeated washing of the semisolid fraction as embraced by (ba) is exemplified in the Example.

In a further preferred embodiment, the method of the first aspect further comprises (d) encapsulating said first composition into a first capsule, wherein preferably (i) said first capsule is a soft capsule; and/or (ii) the amount of said first composition which is encapsulated corresponds to two to ten, preferably five of said olives.

In a further preferred embodiment, the method of the first aspect further comprises (e) encapsulating said second composition into a second capsule, wherein preferably (i) said second capsule is a hard capsule and/or a capsule with an enteric coating; and/or (ii) the amount of said second composition which is encapsulated corresponds to two to ten, preferably five of said olives.

Said second capsule is also designated “capsule 5”, given that it comprises or is obtained from the dried semisolid fraction, or, to the extent said washing (aqueous extraction) of said semisolid fraction has been performed, the dried residue of said aqueous extraction.

In a further preferred embodiment, the method of the first aspect further comprises (f) encapsulating said third composition into a third capsule, wherein preferably (i) said third capsule is a hard capsule; and/or (ii) the amount of said third composition which is encapsulated corresponds to two to ten, preferably five of said olives.

Said third capsule is also designated “capsule A”, given that it comprises said aqueous phase (composition A1) and/or said aqueous extract (composition A2). To the extent use is made of both the aqueous phase and said aqueous extract, which is preferred, said capsule A comprises or is derived from the combined composition A.

Preferably, said third composition, prior to encapsulating it, is absorbed on microcrystalline cellulose.

In more detail, prior to encapsulation, composition A is preferably dried, extracted with a polar organic solvent, and the obtained solvent extract is combined with microcrystalline cellulose.

Preferred polar organic solvents are protic organic solvents such as alcohols, in particular ethanol or isopropanol. Also preferred is that said polar organic solvent is pharmaceutically acceptable. Preference is given to solvents which extract hydroxytyrosol and tyrosol.

After absorbing the solvent extract on cellulose, preferably microcrystalline cellulose, the obtained mixture is preferably dried and subsequently encapsulated.

Preferably said solvent extract and said cellulose are combined in a ratio of 2:3 (w/w).

A more detailed general recipe for producing capsules A is given in Example 1.

In a further embodiment, said method comprises (g) encapsulating said first capsule and said third capsule into a fourth capsule, preferably a hard capsule and/or a capsule with an enteric coating. Such fourth capsule combines beneficial agents comprised in the oil obtained in step (c)(i) with the beneficial agents comprised in the aqueous phase obtained in step (c)(iii). The same applies to any combination of first and third compositions.

In terms of beneficial effects (discussed in more detail below), second composition and capsule, respectively, are less preferred.

The term “enteric coating” has its art established meaning. Accordingly, such coating provides for said capsule to pass the stomach and not be digested prior to reaching the small intestine.

Hard and soft capsules are known in the art. Preferred materials for hard capsules are gelatin and hydroxymethylpropyl cellulose (HMPC).

The present inventors surprisingly discovered that compositions obtained in accordance with the present invention, obtained by the methods of the invention, and being obtained from only about five olives are capable of providing the beneficial effects disclosed further below.

Also, the composition(s) obtained from three, four, six, seven, eight, or nine olives may be used and/or encapsulated as defined above.

It is apparent from the above, that it is envisaged to obtain compositions (or capsules) or combinations of compositions (or of capsules) which on the one hand comprise oil as defined herein above and on the other hand those constituents which are comprised in the aqeuous phase obtained during olive processing in accordance with the methods of the invention.

Not only is surprising that rather small amounts of olives (as stated above, 2 to 10, preferably 5 olives) provide for beneficial effects, but furthermore compliance is improved by administration of capsules to those individuals which reject olives or olive oil for reasons such as the taste thereof not being agreeable. As such, the invention provides for easier and more practical intake of the beneficial compounds comprised in fermented Kalamon olives and the fractions thereof in accordance with the invention.

In a further preferred embodiment, said method comprises prior to step (a), allowing fermentation of said olives to occur, wherein said fermentation occurs (i) for a time span of about 3 to about 12 months, preferably about 4 to about 6 months; and/or (ii) in the presence of autochthonous yeasts, e.g. as they occur on the surface of said olives, wherein preferably no further agent triggering fermentation is added.

Fermentation may also take about 1, about 2, about 4, about 5, about 7, about 8, about 9, about 10 or about 11 months.

In a further preferred embodiment, said Kalamon olives are grown in Peloponnese, preferably in Lakonia or Messinia.

Preferably, said olives are harvested by hand picking. This is a means to ensure that only olives and no leaves are subsequently processed in accordance with the present invention. For the purpose of fermentation, they are preferably placed in brine. Preferred microorganisms catalyzing the fermentation are autochthonous yeasts from the respective region of origin of the olives. Preferred regions of origin are detailed above.

As known in the art, after fermentation, the olives are separated from the brine. They are washed and gently dried. Thereafter the stone is removed.

A preferred preparation procedure consists of the following steps; for further details see the Example.
(1) Olives, processed and debittered using the Kalamata style (see below) are obtained from the fermentation tank, washed and gently dried.
(2) The stone is removed and the flesh is blended/mashed mechanically leading to an olive paste.
(3) The olive paste is centrifuged leading to three distinct phases: Oil (A), semisolid paste (B1) and aqueous phase (C1).
(4) The semisolid paste (B1) is mixed with water and vortexed until homogenization and then filtered. This yields a filtrate (C2) and a semisolid paste (B2). Step (4) may be repeated thereby yielding further filtrates (C3, C4, . . . ).
(5) The aqueous phase obtained after filtration (C2 and, where applicable C3, C4, . . . ) is combined with C1 to yield the final product C.
(6) The semisolid phase B2 obtained after filtration is oven dried yielding the final product B.

The Kalamata style of processing uses black olive fruits of the olive cultivar Kalamon, which are fermented in brine (3-9% salt) for 3-6 months with the autochthonous yeasts found on the surface of the fruit and maintaining a pH value that guarantees lactic fermentation.

In a second aspect, the present invention provides one or more compositions or capsules selected from: (i) a combination of said first and said third composition, both obtained by the method of the first aspect or the fourth capsule obtained by the method disclosed above; (ii) the first composition or capsule obtained by the method of the first aspect; (iii) the second composition or capsule obtained by the method of the first aspect; and (vi) the third composition or capsule obtained by the method of the first aspect.

Particularly preferred are said third composition (composition A), or said third second capsule (capsule A), optionally in conjunction with said second composition (composition S) or said second capsule (capsule S).

Particularly preferred is that said third composition or capsule comprises phenolic compounds such as hydroxytyrosol and tyrosol, wherein preferably each of hydroxytyrosol and tyrosol is present in a mass ratio of 1 mg/g to 20 mg/g. This is generally inherent to the third composition as obtained by the methods of the invention.

Particularly preferred is that said composition A comprises phenolic compounds such as hydroxytyrosol and tyrosol, wherein preferably each of hydroxytyrosol and tyrosol is present in a mass ratio of 1 mg/g to 50 mg/g such as 1 mg/g to 20 mg/g.

Particularly preferred is that said capsule A comprises phenolic compounds such as hydroxytyrosol and tyrosol, wherein preferably each of hydroxytyrosol and tyrosol is present in a mass ratio of 1 mg/g to 25 mg/g such as 1 mg/g to 20 mg/g.

Also particularly preferred is that said second composition or capsule (composition S or capsule S) comprises triterpenic compounds such as maslinic acid and oleanolic acid, wherein preferably each of maslinic acid and oleanolic acid is present in a mass ratio of 0.1 mg/g to 5 mg/g. Having said that, and as stated above, no particular preference is given to said second composition or capsule.

Beneficial effects of olive oil are known from the art, including effects on the lipid metabolism. A common denominator of the prior art is that compounds with antioxidative properties as comprised in olives, in particular phenolic compounds, are held responsible for beneficial effects on lipid metabolism.

The present inventors surprisingly discovered that this is not the case. This has been further explored by allowing phenolic compounds to oxidise and thereafter manufacture the compositions in accordance with the present invention. Also, for such compositions, the beneficial effects as disclosed further below have been confirmed.

Therefore, the present invention relates to a composition comprising all or substantially all ingredients of olives or fractions thereof, said olives being defined as herein above, and said fractions being obtained by the method of the invention as disclosed above, provided that said composition has a significantly lower concentration of phenolic compounds as compared to said olives or is essentially free of said phenolic compounds.

Preferably, said composition or capsule is a food supplement. Preferred food supplements of the invention comprise composition A or capsule A. They may furthermore comprise composition S or capsule S.

A food supplement has to be held distinct from a pharmaceutical composition. For the purposes of the present disclosure, a food supplement is inherently non-therapeutic in nature. Food supplements do not require a doctor's prescription. Rather, to the contrary, they can be purchased by anyone, for example in a supermarket.

The compositions of the present invention may also be used for medical purposes. As such, the present invention also relates to the use of the above disclosed fractions, compositions or capsules for use in medicine. Related thereto, provided are pharmaceutical compositions comprising or consisting of said fractions, compositions or capsules. Preferred is composition A or capsule A, optionally combined with composition S or capsule S.

The fractions, compositions and capsules are particularly useful for treating dyslipidemia. Dyslipidemia is a disorder of lipoprotein metabolism, including lipoprotein overproduction or deficiency. Dyslipidemias may comprise elevation of the total cholesterol, the low-density lipoprotein (LDL) cholesterol and the triglyceride concentrations, and a decrease in the high-density lipoprotein (HDL) cholesterol concentration in the blood.

Dyslipidemia comes under consideration in many situations including diabetes, a common cause of hyperlipidemia. For adults with diabetes, it has been recommended that the levels of LDL, HDL, and total cholesterol, and triglyceride be measured every year. Optimal LDL cholesterol levels for adults with diabetes are less than 100 mg/dL (2.60 mmol/L), optimal HDL cholesterol levels are equal to or greater than 40 mg/dL (1.02 mmol/L), and desirable triglyceride levels are less than 150 mg/dL (1.7 mmol/L).

In line therewith, and in a third aspect, the invention provides such fraction, composition or capsule for use in a method of treating (a) dislipidemia; (b) disorders associated with elevated levels of LDL cholesterol; (c) disorders associated with elevated levels of total cholesterol; and/or (d) disorders associated with low levels of HDL cholesterol. Deviant levels (“elevated”; “low”) are defined with respect to the desired values given above.

In a preferred embodiment, (a) said levels are concentrations in serum, blood or plasma; (b) said elevated levels of LDL cholesterol are serum concentrations, preferably between about 200 mg/dl and about 300 mg/dl; (c) said elevated levels of total cholesterol are serum concentrations above about 200 mg/dl serum, preferably between about 200 mg/dl and about 300 mg/dl serum, more preferably between about 215 mg/dl and about 275 mg/dl serum; and/or (d) the individual to be treated does not receive any further medication for the treatment of hypercholesterolemia such as statins.

The term “about” in the context of this invention refers to deviations from the recited values as they are commonly observed in the art, such as in the context of blood analysis. Exemplary deviations are +/−20%, +/−10% and +/−5%.

In a further preferred embodiment of medical uses in accordance with the present invention, said disorders are selected from cardiovascular diseases, atherosclerosis, cardiac infarct, stroke, thrombosis and embolism. Also included is low to moderate cardiovascular risk, preferably heart scores below 5%. “Score” in this context refers to Systematic COronary Risk Evaluation (SCORE): high and low cardiovascular risk charts based on gender, age, total cholesterol, systolic blood pressure and smoking status, with relative risk chart, qualifiers and instructions.

Related to the food supplement in accordance with the fourth aspect, the present invention provides, in a seventh aspect, the use of the food supplement of the invention for lowering (a) LDL cholesterol; (b) lowering total cholesterol; and/or (c) increasing HDL cholesterol in an individual.

As noted above, (a) said use is non-therapeutic; and/or (b) said individual does not suffer from and preferably is not at risk to develop any of the disorders defined herein above.

The administration of compositions of the invention does not trigger significant changes in serum glucose transaminases, urea and creatinine. Altogether, there is no indication that consumption of compositions in accordance with the present invention would have side effects.

Regardless of whether the use is therapeutic or non-therapeutic, a preferred daily dosage is one capsule. As noted above, one capsule preferably comprises material obtained from 2 to 10, preferably from 5 olives. As a consequence, and to the extent compositions in accordance with the present invention are not formulated into capsules, preferred dosages are such that daily administration of the material derived from 2 to 10, preferably 5 olives is achieved.

As regards the time period of administration, there are no particular limits. To the contrary, life long intake is possible and recommended. Beneficial effects occur after an administration for about one, two or three months.

Generally speaking, the above suggested dosages and dosage regiments will not require modification depended on age, sex, weight etc. of the individual to whom compositions are to be administered. Having said that, and to the extent such modifications are nevertheless perceived as being adequate, they can be done by the attending doctor or physician (in case of medical uses) without further ado. As noted above, an amount of compositions of the invention corresponding to about 5 olives is preferred.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The examples illustrate the invention.

### EXAMPLE 1

### Manufacture of Compositions of the Invention

50 olive fruits are washed and dried gently before weighing. Then, the stones are separated from the flesh and the flesh is weighed.

### Weighing Results:

whole olive fruits: 253.5 gr (average weight of each fruit: 4.71 gr)
50 olive stones: 44.4 gr
Fruit flesh from 50 olives: 201.0 gr

After weighing, the flesh is mashed using a blender until an olive paste is obtained. The paste is then centrifuged for 10 min at 4.000 rpm. After centrifugation, the olive paste is found separated into 3 layers (phases), i.e., aqueous phase, lipid phase and a semisolid paste in the middle.

The upper layer consists of olive oil, which is analyzed using the protocol described in Karkoula et al., Journal of Agricultural and Food Chemistry, 2012, 60, 11696-11170. The total weight is 13.5 gr. Olive oil analysis shows the presence of tyrosol and hydroxytyrosol in relatively low concentrations. “Low concentrations” in this context refers to concentrations relative to those in olive flesh as well as to those in aqueous extracts according to the invention.

The olive fruit paste stays in the middle layer of the 3-layered material as obtained after the centrifugation. By gently scraping the surface, any olive oil residues that may be present on the upper side of the paste are removed. Then, the paste is stored separately from the liquid phases for further processing as described below. Olive fruit paste after the centrifugation, total weight: 151.3 gr

The lower phase of the 3-layered material consists of the aqueous phase originally comprised in the olive flesh. Total weight before drying: 22.7 gr The olive fruit paste is divided into 50 mL falcon tubes and equal amount of distilled water to the amount of fruit paste is added in each of them. For resuspending, a heavy duty vortexer is used, for 1 min, until homogenization. The mixture is filtered using paper filter. This step is repeated with fresh water for two more times. All the water from these extractions, including the water from the step of centrifugation are pooled together and filtered once again.

The combined aqueous phases are dried under vacuum using a rotary evaporator, and then weighed. Total weight of dry extract: 6.25 g. In order to quantify the tyrosol, hydroxyl tyrosol and lactic acid content, we use syringaldehyde as internal standard and qNMR. The NMR spectrum indicates that this extract contains 7 mg/g tyrosol, 9 mg/g hydroxytyrosol and 51 mg/g lactic acid. No maslinic acid is detected.

To enrich the dried aqueous extract, remove inorganic material (e.g. sodium chloride) and prepare the material for encapsulation, the following procedure is applied: The dry aqueous extract is dissolved in ethanol or isopropanol (1:5 w/v) and the insoluble part is discarded by filtration. The ethanol solution is mixed with microcrystalline cellulose (2:3 w/w dry weight) and dried under vacuum using a rotary evaporator affording a powder that is encapsulated in hard capsules. Each hard capsule has a content of 500 mg including 6 mg tyrosol, 10 mg hydroxytyrosol and 51 mg lactic acid. The preferred daily dose is equivalent to 5 olives is contained in two capsules.

After the three water extractions as described above, the olive fruit paste weighs 146.3 g. The material is dried using an oven at 60° C. for 72 hours, with gentle stirring every 24 hours. After 3 days the dry weight of the product is 46.5 g. The dry product, similar to a cookie, is pulped using a blender to give a semi-liquid texture resembling a jam or cream. In order to quantify the phenolic content of this material, we use a methanol:water extraction, followed by defatting using cyclohexane and internal standard addition. Using qNMR and data processing, the results show that this product contains 0.24 mg/g tyrosol and 0.54 mg/g hydroxytyrosol, as well as 3.81 mg/g lactic acid and 3.23 mg/g maslinic acid.

1. A method of producing at least one composition, said method comprising:
(a) preparing a paste from flesh of fermented Kalamon olives;
(b) subjecting said paste to a separation process yielding oil, a semisolid fraction, and an aqueous phase;
(c)
(i) harvesting said aqueous phase, thereby obtaining a composition A; and
(ii) optionally, drying said semisolid fraction, thereby obtaining a composition S.

2. The method of claim 1, further comprising:
(ba) washing said semisolid fraction obtained in (b) with water, thereafter combining said water with said aqueous phase obtained in (b), and thereby obtaining a washed and dried composition S of (c)(i), and a combined aqueous phase yielding composition A of (c)(ii).

3. The method of claim 1, further comprising:
(d) encapsulating said composition A into a capsule A, wherein
(i) said capsule A is a hard capsule; and
(ii) an amount of said composition A which is encapsulated in said capsule A corresponds to two to ten of said fermented Kalamon olives.

4. The method of claim 1, further comprising:
(e) encapsulating said composition S into a capsule S, wherein
(i) said capsule S is a hard capsule or a capsule with an enteric coating; and
(ii) an amount of said composition S which is encapsulated in said capsule S corresponds to two to ten of said fermented Kalamon olives.

5. The method of claim 1, further comprising, prior to step (a), allowing fermentation of said olives to occur, wherein said fermentation occurs
(i) for a time of about 3 months to about 12 months; and/or
(ii) in the presence of autochthonous yeasts.

6. The method of claim 1, wherein said Kalamon olives have been grown in Peloponnese.

7. One or more compositions selected from:
(i) composition A; and
(ii) composition S,
wherein each of said composition A and said composition S is obtained by the method of claim 1.

8. The one or more compositions of claim 7, wherein
(i) said composition A comprises phenolic compounds hydroxytyrosol and tyrosol in a mass ratio of 1 mg/g to 50 mg/g;
(ii) said capsule A comprises phenolic compounds hydroxytyrosol and tyrosol, in a mass ratio of 1 mg/g to 25 mg/g; and/or
(ii) said composition A comprises lactic acid in an amount less than or equal to 150 mg/g.

9. The one or more compositions of claim 7, wherein said composition S comprises triterpenic compounds maslinic acid and oleanolic acid in a mass ratio of 0.1 mg/g to 5 mg/g.

10. A food supplement comprising the one or more compositions of claim 7.

11. A method of treating any one of:
(a) dyslipidemia;
(b) disorders associated with elevated levels of LDL cholesterol;
(c) disorders associated with elevated levels of total cholesterol; and/or
(d) disorders associated with low levels of HDL cholesterol,
comprising administering the composition of claim 1 to an individual to be treated.

12. The method of claim 11, wherein
(a) said levels of LDL cholesterol, total cholesterol and HDL cholesterol are concentrations of LDL cholesterol, total cholesterol and HDL cholesterol in serum, blood or plasma;
(b) said elevated levels of LDL cholesterol are serum concentrations;
(c) said elevated levels of total cholesterol are serum concentrations above about 200 mg/dl serum; and/or
(d) the individual does not receive any further medication for the treatment of hypercholesterolemia.

13. The method of claim 11, wherein said disorders are selected from cardiovascular diseases, atherosclerosis, cardiac infarct, stroke, thrombosis and embolism.

14. (canceled)

15. (canceled)

16. The method of claim 1, wherein said separation process is centrifugation.

17. The method of claim 5, wherein said fermentation occurs for a time of about 4 months to about 6 months.

18. The method of claim 5, wherein said fermentation occurs in the presence of autochthonous yeasts and no further agent triggering fermentation is added.

19. One or more capsules A obtained by the method of claim 3.

20. One or more capsules S obtained by the method of claim 4.

21. A food supplement comprising the one or more capsules of claim 19.

22. A food supplement comprising the one or more capsules of claim 20.
